(19) United States
(12) Patent Application Publication
(10) Pub. No.: US 2025/0356287 A1
(43) Pub. Date: Nov. 20, 2025

(54) COMPREHENSIVE CONFIDENTIAL INFORMATION MANAGEMENT AND COMMUNICATION

(71) Applicant: Justin O'Keefe, Biloxi, MS (US)

(72) Inventor: Justin O'Keefe, Biloxi, MS (US)

(21) Appl. No.: 19/284,246

(22) Filed: Jul. 29, 2025

Related U.S. Application Data
(63) Continuation-in-part of application No. 17/824,499, filed on May 25, 2022, now abandoned.

(51) Int. Cl. G06Q 10/0631; G06F 21/32; G06F 21/62; G06Q 50/18

(52) U.S. Cl. CPC G06Q 10/06315; G06F 21/32; G06F 21/6245; G06Q 50/186

(57) ABSTRACT

A comprehensive confidential information management and communication system and method to gather, maintain, update, and cross-reference information and instructions pertinent to a person's health, social, and financial well-being during later years, and implementation of plans for possible incapacity and for eventual death, coordinating with various professional service providers each having an independent professional relationship with the person, and timely securely conveying appropriate information and instructions to the appropriate professionals upon changes of conditions or instructions. The comprehensive confidential information management and communication system provides a rules-relationship database, an information database, a system server with processing circuitry and memory, an access controller, deep learning model analysis of each person's information, rules, and relationships, deep learning model analysis and update of outside rules and information, authorizing use of the system, updating by the person of the rules-relationships database and the information database, updating by a professional of the information database, and secure means for a professional to receive relevant updated information from the information database.

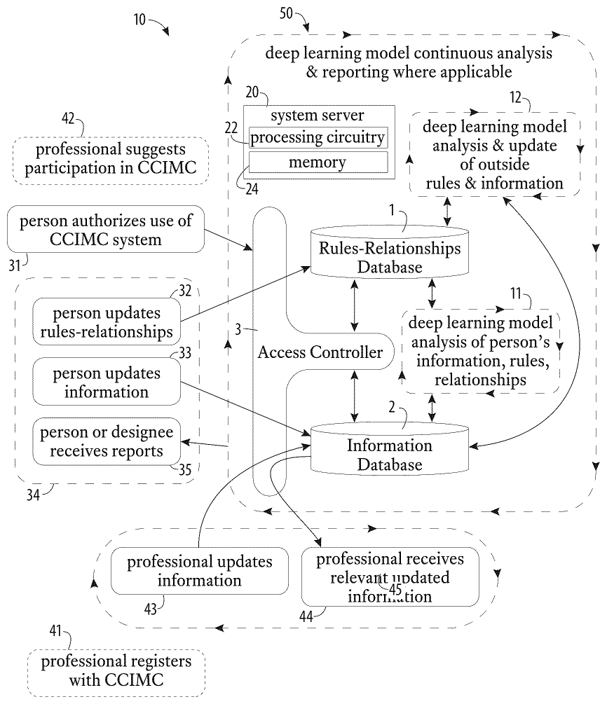

### CROSS-REFERENCE TO RELATED APPLICATIONS

This application is a continuation-in-part of co-pending application Ser. No. 17/824,499, filed on May 25, 2022, which is a continuation-in-part of U.S. application Ser. No. 16/128,814, filed on Sep. 12, 2018, the full disclosures of which are incorporated by reference herein and priority of which are hereby claimed.

### BACKGROUND

This invention provides a comprehensive confidential information management and communication system and method to gather, maintain, update, and cross-reference information and instructions pertinent to a person's health, social, and financial well-being during later years, and implementation of plans for possible incapacity and for eventual death, coordinating with various professional service providers each having an independent professional relationship with the person, and timely securely conveying appropriate information and instructions to the appropriate professionals upon changes of conditions or instructions.

The “person” under consideration is a person who wants to make plans and provisions for eventual death and for possible periods of partial or total incapacity to make and implement decisions. The person might have obligations and interests with other persons, including family, might be actively involved in running a business or managing investments, might have present or future benefits such as retirement, trusts, or annuities, might have vested, unvested, or contingent interests, and might anticipate an inheritance. The person might occasionally or regularly use the services of service providers such as legal or financial advisors, and institutions such as banks and hospitals.

Typically, the laws of the jurisdiction where a person is domiciled at the time of death, and possibly of a different jurisdiction where real property is located, will most control the administration of that person's estate. But a person might reside in a care facility or reside with family or others in a different jurisdiction for some time before their death, or might be evacuated or transferred to a different jurisdiction because of a disaster, and then might die within a jurisdiction different from the one anticipated. Different jurisdictions have different laws regarding death and inheritance, with differences ranging from subtle to drastic. For example, although the laws of most jurisdictions prevent implementation of instructions, qualifications, or criteria which would continue to affect the ownership of private property for a time long beyond the death of a particular person, such as various implementations of the rule against perpetuities which usually requires that all interests must vest not later than 21 years after the end of some life in being at the creation of the interest, some jurisdictions, including some US states, have changed their laws, usually their trust laws, to allow significant increases in time. Also, some jurisdictions seek to prevent transfers of assets at death outside of the court succession process, while other jurisdictions allow such bypassing. Therefore, the specific effects of any specific jurisdiction's laws must be taken into account by persons planning and organizing an estate. The planning will usually be done with the help of a legal advisor qualified in the field and admitted in the relevant jurisdiction, and, for all but simple circumstances, with a financial advisor and probably with other professionals.

Although the law, the courts, and the last will and testament will ultimately control the final disposition of a deceased person's estate, the law moves very slowly, by design, and the courts are unlikely to even be given notice of a person's death until many weeks after the death, when a death certificate is available. In the meantime, a funeral must be planned and held, a cremation might have to be authorized, people and institutions must be notified, bills must be paid, and pets must be fed. If the deceased person was actively involved in a business or was actively managing investments, the last will and testament might or might not provide adequate plans or instructions, and in any case will take far too long to implement. For a business or active investments, the incapacity of a person might have the same or even worse consequences, the last will and testament will probably not be activated, and the will might not have any provisions addressing incapacity. Many significant aspects of a person's life, possible incapacity, and the period of weeks after death cannot be timely and adequately managed through the last will and testament. And if the provisions of the will are not even known to the people and institutions having to make decisions and take actions during incapacity and immediately after the death, then there is a possibility that the decisions and actions will be later found to be in conflict with whatever provisions might be in the will.

Among the significant unknowns facing a person trying to formulate an end-of-life plan are whether, to what extent, and for how long the person might become incapacitated before death. If a person is active in managing a business or actively managing changes to investment portfolios then a more comprehensive plan for that person should include provisions such as creating instructions or guidelines for continued management if the person becomes incapacitated, or for converting those assets to assets requiring no active management.

In most jurisdictions, the handling of the bodily remains of the person will be done by a funeral director service provider, who will submit information and notification to the appropriate governmental entity, which will then create a death certificate and issue certified copies of that death certificate. In most jurisdictions, the issuing of a death certificate is a necessary prerequisite to the initiation or the completion of most of the processes of administering and settling the person's estate. For example, the lawyer cannot obtain any settlement of the estate from a court without a certified copy of the death certificate. The death certificate is issued by a government bureaucracy and will take some time to issue. Most financial institutions require a death certificate before releasing funds needed to pay immediate expenses and to administer the estate. Different institutions and organizations have different policies about whether a certified death certificate or just a photocopy will be accepted.

Persons wanting to plan for eventual death do not know precisely when nor precisely how death will occur, and do not know whether death will be preceded by a significant period of diminished capacity or incapacity, or how long or how expensive such a period might be. The person therefore does not know, for example, what amount of assets should be held in reserve against potentially large expenses, as opposed to doing things of interest to the person, such as travel, continued learning, or speculative ventures, or to share some of those assets to provide timely immediate help to other persons or institutions.

Persons planning for advanced age and eventual death and possible incapacity use and rely on the services of many service providers. The terms “profession” and “professional” without explicit qualification are used here to cover the traditional professions of law, medicine, education, and religion, and to cover persons, organizations, and institutions providing relevant services such as funeral directors, brokers, bankers, insurers, financial advisors, hospitals, and organizations providing housing, transportation, and in-home services. Each profession or professional is usually regulated by one or more governmental or self-governing entities. Each professional generally has a direct relationship with and responsibility to the person, often including a direct duty of confidentiality which can vary in scope among the various professions.

The various professions and professionals serving a person will have unique areas of primary concern and will develop unique sets of information, often confidential information. But some of the information developed by one professional ought to be made available to certain other professionals in order to best serve the client. In some cases, good service to the client could be harmed by the failure to provide information developed by one professional to other professionals. For example, the primary function of a last will and testament is instructions for disposition of a person's estate, which is not usually put into effect until at least several days after the person dies, and is put into effect by a court, with the involvement of lawyers who might or might not have any knowledge about the specific provisions of the will until at least several days after the person dies. But the last will and testament might also include a statement of the person's instructions for the disposition of remains, which is information that the funeral director needs to know either prior to or immediately after the person's death. The last will and testament might also contain living-will instructions about health-care matters where the instructions necessarily must be made known to healthcare professionals before the person's death. But only the relevant provisions need to be disclosed to the funeral director and healthcare professionals, who do not need to know the provisions for distribution of the estate.

Senior-living facilities exist which provide both housing and healthcare in an integrated way, such as providing for independent living with provisions for transitioning to assisted living and to skilled nursing if and when needed. A person living in such a facility would likely have physician-patient relationships with several outside healthcare professionals, who would need to exchange information with in-house healthcare staff. The person is best served when the outside healthcare professional can communicate instructions to and can receive compliance and other observations from in-house professionals. Some senior-living facilities operate under an arrangement where an entrance fee of several hundred-thousand dollars is collected, and is 90% refundable to the person or the person's estate, plus monthly fees of thousands of dollars. Under such an arrangement the combined residential and healthcare facility also functions as a financial institution which might be holding the greatest asset of a person's estate, which is information that other professionals serving the same person might need to know.

In the US, the Health Insurance Portability and Accountability Act of 1996 (HIPAA) governs health-insurance and therefore substantially all healthcare information. The HIPAA Privacy Rule provides national regulations for the use and disclosure of Protected Health Information (PHI) in healthcare treatment, payment, and operations by covered entities. In 2013, HIPAA was extended to cover business associates. Almost any professional serving a person of advanced age in any capacity can be said to be dealing with health information, and the professional either must learn the HIPAA rules, with changes and with developing interpretations, or must stop sharing information. Although the “P” in HIPAA stands for portability, not privacy, the effect of the law has been great concern about privacy requirements. And concerns about privacy seem to be obstructing the goals of portability. The threat of HIPAA privacy-violation penalties can only make professionals less likely, not more likely, to share information.

Therefore, information concerning a person, which has been developed by various professionals serving that person, is being kept separate and secret in silos of information, which operates to the detriment of the person's interests. Vital documents or collections of information are being kept entirely secret by one professional out of an abundance of caution, depriving another professional, who also has a confidential relationship with the person, of relevant information which might only be a subset of, or might only be incidental to the document or collection of information.

The term “survivors” is used broadly to encompass other persons, organizations, or institutions who are somehow affected by or interested in a person's death or incapacity, including heirs, relatives, next-of-kin, spouses, children, or non-family persons, affiliated businesses, educational, or religious institutions.

At a person's death, almost all of the separate threads of the person's life, history, relationships, and involvement are brought forward in importance and relevance. Whatever unresolved and undecided matters that a person leaves either become unimportant or become critically important. Ideally, complete information relevant to each survivor should be made available to the survivor either prior to or immediately upon the person's death because many actions have to be taken very shortly after the death. In some circumstances, such as the person having continued active control of a business or active management of an investment, incapacity of the person can create similar needs for the availability of relevant information and communication and implementation of whatever relevant plans and instructions the person may have created. If a person becomes incapacitated for a significant amount of time, without adequate plans and provisions already in place, then potential gains and benefits might not be realized, and losses and penalties might be suffered. Even for a person fully retired, some ongoing monthly bills must be paid, people, organizations, and institutions must be notified, and expenses incident to the death or incapacity must be met.

When a person grows old, becomes incapacitated, or approaches death, the person's circumstances can change in ways that might affect when and how certain contingent elements or phases of the person's plans should be implemented. For example, a person might have instructed a financial professional that upon the person's health reaching a certain threshold of decline, assets and financial dealings should be rearranged in a certain way. But the person whose health has declined might not be able to personally inform the financial professional of the decline. The person's health professionals will know of the decline immediately, and will have a very well-informed knowledge of the present state of the person's health and the prognosis for the immediate and longer-term future. But at present, there is no system for both protecting the confidentiality of the person's total health information while sharing a relevant subset of that information with another professional who also has a confidential relationship with the person, for the purpose of implementing the person's stated plans and instructions.

A deep learning model can autonomously ‘learn’ the importance or significance of an observation as a feature to be incorporated into the analysis. This autonomous ‘learning’ includes analysis of combinations of observations, and reference to cumulative learning obtained from all prior observations and the known actual outcomes from those prior instances.

In a simplified example, where x is an observation or feature such as information of an occurrence such as an accidental injury, a medical event, or a test result, the deep learning model generates Y as an indicator of probability of some outcome or outcomes using, at various layers, either a generative model of the joint probability distribution or a discriminative model of the conditional probability, in the process detecting, classifying, and ‘learning’ the features that can be found in the information.

What is needed is a comprehensive confidential information management and communication system and method providing secure management of confidential information about a person, application of a deep learning model to the individual and the aggregated information, and the timely secure communication of relevant information according to the person's stated instructions either with the person, the person's designees, or among various professionals who each have a confidential relationship with the person, for the purpose of implementing the person's overall plans and instructions.

US Publication No. 2016/0283877 for a “Death Care Management System and Method,” as published on Sep. 29, 2016 by inventors G. Scott Mindrum et al., discloses a system that provides automated and semi-automated tools and methods that may be used by providers in the death care industry to manage time sensitive tasks and share information with third party providers of goods and services.

US Publication No. 2015/0242814 for “Systems and Methods for Handling Social Digital Accounts and Assets upon Death or Incapacitation,” as published on Aug. 27, 2015 by inventor Rana A. Saad, discloses a system to allow users to specify how they would like to treat their digital assets embedded in their online and social accounts upon their death, by choosing one or a mix of numerous options for online verification and confirmation of a person's death by comparing against the online death records of all the States; and a system and protocol for second level verification of the death from the next-of-kin or a person designated by the user to be passed on to the beneficiary.

US Publication No. 2015/0019449 for a “Method to Transfer Personal Financial Information and Other Hard to Replace Documents to a Selected Recipient Post Death,” as published on Jan. 15, 2015 by inventor Navin Murli Lalwani, discloses a safe, secure, and reliable process that allows a family member to select one or more loved ones who should be able to receive all the financial details, hard to replace documents, and other information of a person in case of an unfortunate sudden death. The system and methods surrounding this process can help avoid the various follow ups and errands that might be required with various agencies, such as banks, creditors, tax agents, insurance companies, and the like, to find all of the related and necessary information. More specifically, a new user can register (create a profile) himself using the GUI and updates all information. The user can update all information for each section, including assets, liabilities, and document uploads. The data can cover practically any data the user wishes to store. Specific examples of profile data include user details, user contact details, membership details, and user preference settings. Specific examples of asset data include bank accounts, insurance policies, fixed deposits, trading accounts, stocks, bonds, retirement accounts, collectibles, art, jewelry, furnishings, and the like. Specific examples of liabilities include credit cards, direct debit payments (such as utility bills, phone bills, car loan payments, homeowner's association fees, mortgages, magazine subscriptions, life insurance premiums, health insurance premiums, newspaper subscriptions, and the like), and loans (such as home loans, car loans, student loans, and the like). Specific examples of data uploads include important documents such as wills, property deeds, birth certificates, company documents, important bills, passport copies, and other hard to replace documents. Specific examples of information under the information recipients tab can include name, relationship, email, phone number, status (such as confirmed, pending, or the like), test a report (to test run a report that will be sent to the beneficiary), and action (to edit or delete information). The user can then select one or more information recipients. With this function the registered user can select multiple individuals who can receive the information after his or her death. The user can enter the first name, last name, email, and phone number of the person he wishes to select as an information recipient. An email can be sent out to that selected information recipient requesting to “Accept” or “Reject” the information recipient role. If the selected information recipient “Accepts” the request, then he/she shall be navigated to the new screen to create a 4-digit security PIN, two security questions, and confirm date of birth. Once completed, an email confirmation is sent to the registered user notifying that the information recipient has confirmed the request and an email is sent to the information recipient which describes the steps the information recipient needs to take when claiming all the information. After the death of the user, an information recipient can submit a request for registered user's information. The information recipient can navigate to the website homepage (or access appropriate software and/or applications, or the like) and click on the “File a Claim” link to claim information of registered user.

US Publication No. 2017/0132736 for a “Deceased Notification System and Method,” as published on May 11, 2017 by inventor William Hampton Switzer, Sr., discloses a deceased notification system utilizing hub and spoke architecture to facilitate notifications to multiple organizations. By having all disparate and disjointed company deceased notification processes and their corresponding required documentation self-contained in one system, the invention provides a means for those settling estates to utilize a “hub and spoke” model to provide deceased notifications, whereby they register someone's death at the hub, and have the system proactively push deceased notifications to all pertinent organizations at the earliest time. This will save all parties time, provide quicker notifications to prevent deceased identity theft, as well as the stoppage of charges for services that are no longer needed by the decedent.

US Publication No. 2008/0163342 for a “System and Method for Managing Information Relating to End-of-Life Issues,” as published on Jul. 3, 2008 by inventor David L. Christopherson, discloses a system and method for securely and centrally storing, updating, and transferring information pertaining to the end-of-life issues of an administrator. The information can be input into the system by an administrator or by the administrator's representative. The information is input and stored within topical folders and sub-folders, making the information easy to locate again in the future by the representative.

US Publication No. 2003/0182290 for an “Integrated Life Planning Method and Systems and Products for Implementation,” as published on Sep. 25, 2003 by inventor Denise S. Parker, discloses a method for facilitating final estate planning that comprises gathering data in response to a series of questions organized into four points of estate planning, which are (1) client assets; (2) client fiscal and human/pet care responsibilities (i.e. creditors, children, spouse, employers, and others that are dependent upon the client); (3) desired disposition of the client's body before (e.g., assisted living, hospice, palliative care, life support) and after death (corporal disposition); and (4) funding arrangements and documentation for asset disposition to cover fiscal, human/pet care responsibilities, living body and subsequent remains disposition, and to carry out the client's other wishes. A system for performing the method includes a computer program that is preferably accessible via an Internet web site. A unique identification card, known as a denicard™, is provided which includes information for accessing the bearer's or owner's end of life plans and data that is stored in a database accessible via the web site or a designated entity, wherein the stored data is generated by the system when the financial estate planning method was performed.

US Publication No. 2018/0137585 for “Estate Planning and Administration,” as published on May 17, 2018 by inventor Marius Van Niekerk, discloses an estate management platform for administering estate data of a person, which includes a remote access interface for receiving incoming data connections, the remote access facility having secure data access facility and being operable to access an estate database and an estate database, including a predefined structure of estate categories for storing estate data of a person in predefined estate categories.

### SUMMARY OF THE INVENTION

This invention provides a comprehensive confidential information management and communication system and method to gather, maintain, update, and cross-reference information and instructions pertinent to a person's health, social, and financial well-being during later years, and implementation of plans for possible incapacity and for eventual death, coordinating with various professional service providers each having an independent professional relationship with the person, and timely securely conveying appropriate information and instructions to the appropriate professionals upon changes of conditions or instructions.

The comprehensive confidential information management and communication system applies a deep learning model capable of predicting an outcome by analyzing information about an individual by, among other steps, analyzing multiple observations about the individual in light of a model built from past observations of the same and of different individuals, and the known outcomes corresponding to the past observations. The deep learning model analysis is done substantially continuously, and the new observations are added to the model system. When a prediction is made the prediction and the details supporting the prediction are also added to the model system for the purpose of tracking and improving operations.

The disclosed system provides a comprehensive, computer-implemented platform for managing and communicating confidential information related to an individual's health, social, and financial well-being. The system comprises a system server/processor having processing circuitry and memory configured to store a rules-relationship database containing user-defined rules and relationships among various data elements, and an information database that maintains dynamic records of the individual's status, directives, and associated metadata.

An access controller operationally connected to the system server governs secure entry to the system, authenticating users and authorizing access based on predefined roles and relationships. The system employs a deep learning model configured to analyze the individual's data, rules, and relationships to generate predictive insights. The model is trained on historical data from the individual and other anonymized individuals, and continuously updates itself with new observations and prediction outcomes.

The system also includes a module for analyzing external rules and information, allowing the deep learning model to incorporate evolving standards, regulations, and third-party data. Users may update the rules-relationship database and the information database through a secure interface, while authorized professionals may contribute updates to the information database. Upon detecting a change in the individual's condition or directives, the system identifies relevant professional service providers and securely transmits updated information to them. All transmissions are logged for audit and compliance purposes.

The comprehensive confidential information management and communication system provides a rules-relationship database, an information database, an access controller, deep learning model analysis of each person's information, rules, and relationships, deep learning model analysis and update of outside rules and information, authorizing use of the system, updating by the person of the rules-relationships database and the information database, updating by a professional of the information database, and secure means for a professional to receive relevant updated information from the information database.

### BRIEF DESCRIPTION OF THE DRAWING

A drawing is provided showing a diagram of the comprehensive confidential information management and communication system and method of the invention.

### DETAILED DESCRIPTION OF THE INVENTION

A diagram of the comprehensive confidential information management and communication system and method 10 is shown in the drawing.

The comprehensive confidential information management and communication system and method 10 provides secure management of confidential information about a person who wants to make plans and provisions for eventual death and for possible periods of partial or total incapacity to make and implement decisions, and the timely secure communication of relevant information according to the person's stated instructions among various professionals who each have a confidential relationship with the person, for the purpose of implementing the person's overall plans and instructions. More detailed characterizations of the person and the professionals, and several illustrative examples of needs for timely secure communication of relevant information among various professionals, are treated above.

The comprehensive confidential information management and communication system 10 provides a deep learning model continuous analysis and reporting where applicable 50, providing a deep learning model of machine learning using multiple layers of classification, regression analysis, and feature learning or representation learning, known in the artificial-intelligence field. The comprehensive confidential information management and communication system 10 does not merely apply pre-defined algorithms to pre-defined data sets, but iteratively recognizes, classifies, and ‘learns’ properties termed as features, ‘learns’ about the possible correlations among features and outcomes, and ‘learns’ further about possible causal relationships, iteratively developing, testing, and revising a very strong predictive model of the correlations of single or multiple interacting features and outcomes. The deep learning model of the comprehensive confidential information management and communication system 10 has access to a large amount of information from a large number of individuals, and has ongoing access, allowing predictions to be compared with actual outcomes for individuals and groups, allowing the deep learning model to correct and refine its feature detection and modeling. The deep learning model runs substantially continuously, not only updating the data, but also updating the classifications, regression analyses, and feature learning of the model itself.

Implementation of the comprehensive confidential information management and communication system and method 10 requires several tightly integrated interdependent modules of highly secure custom software or computer code. These custom software modules can utilize available standard secure communications and database management back-end subsystems and can be implemented on standard computer and communications equipment having sufficient capacity and security.

The comprehensive confidential information management and communication system 10 provides a rules-relationships database 1 which stores records about the relationships between the person and various professionals, such as the person's lawyer or lawyers, the person's financial advisor or advisors, or the person's funeral director under a pre-planning arrangement, and which stores records about the rules set by the person allowing or disallowing access to specific information or categories of information for each professional or category of professional, including rules which are contingent upon some event or some condition or circumstances, including but not limited to death or incapacity of the person. The rules-relationships database 1 can also store information about relationships among people, for example among spouses, family members, or business or personal partners, including relationships such as co-ownership, established by law or by agreement.

The comprehensive confidential information management and communication system 10 provides an information database 2 which stores records of information about the person, at various levels of granularity or detail. For example, a record might indicate that the person has a will, but with no further details, while another record might indicate that the person's will contains instructions for disposition of the person's remains, and another record might contain the details of the person's instructions for disposition. The information in the information database 2 includes but is not limited to information about businesses, investments, annuities, sources of income, rents, or royalties, assets and liabilities, existence of documents such as contracts or agreements, details of document content, or copies of documents. The information database 2 can also contain information about a person's status, including whether the person has died, has become incapacitated, or less dramatic changes such as retirement or change of marital status.

The comprehensive confidential information management and communication system 10 provides an access controller 3 which controls access to the information stored in the information database 2 in accord with the rules and relationships stored in the rules-relationships database 1. The access controller functions as a communication module supporting encrypted messaging and audit trails for compliance with privacy regulations. The access controller 3 verifies that an attempted access is being made by either a person or a professional registered with the system. If access is made by a professional potentially representing more than one person, the professional's representation of a specific person is identified and verified. The access controller 3 will allow the person to initially enter or to update the rules and relationships and other information, as treated below. The access controller 3 will allow a professional to access the person's information as authorized, and to update information as authorized, as treated below.

The system 10 can be configured to perform various functions in accordance with one or more embodiments or examples described herein. Thus, the system 10 can provide means for implementation of mechanisms, techniques, processes, functions, components, systems described herein and in particular compilation and optimization of deep learning models 11 and 12. The system 10 can include a general-purpose processor or specially designed circuits to implement various functions, components, or processes described herein in various embodiments.

The system 10 includes a system server or main processor 20 comprising a processing circuitry module 22 and a memory module 24. The system server 20 is operationally connected to the access controller 3 and is configured to perform the functions and processes described herein in combination with software or without software. The memory module 24 functions as a secure data repository configured to store information and instructions relating to the individual's health status, familial, business, and professional relationships, financial assets, and legal directives.

The processing circuitry module 22 functions as a data processing module configured to gather, maintain, update, and cross-reference the information and instructions from multiple sources stored in and added to, the memory module 24. The data processing module 22 utilizes machine learning algorithms to identify inconsistencies or gaps in the stored information. The processing circuitry comprises a coordination engine configured to identify, and associate the stored information and instructions with a plurality of professional service providers, each having an independent relationship with the individual. The processing circuitry 22 comprises a condition monitoring module configured to detect changes in the individual's status or instructions.

The system server 20 functions, inter alia, as deep learning processor configured to train a classification module residing within the server 20, to make predictions for each of multiple combinations of the health metrics, rules and relationship data based on the learned features of the training algorithms. The processor 20 is also configured to train machine interpretable deep learning models 11 and 12 to make predictions within the network based on the output of the classification module. The access controller 3 comprises an output module configured to present the person with the prediction of the outcome based on the analysis of health metric, rules, and relationship datasets, through a user interface. The output module is further configured to present an explanation of the prediction, including identifying the features that contributed most significantly to the prediction.

In various examples, the processing circuitry 22 can be a digital signal processor (DSP, an application-specific integrated circuit (ASIC), programmable logic devices (PLDs), field programmable gate arrays (FPGAs), digitally enhanced circuits, GPU, CPU, or comparable device or a combination thereof. In some examples, the processing circuitry 22 can be a central processing unit (CPU) configured to execute program instructions to perform various functions and processes described herein. Accordingly, the memory 24 can be configured to store program instructions. The processing circuitry 22, when executing the program instructions, can perform the functions and processes. The memory 24 can further store other programs or data, such as operating systems, application programs, and the like. The memory 24 can include non-transitory storage media, such as a read-only memory (ROM), a random-access memory (RAM), a flash memory, a solid-state memory, a hard disk drive, an optical disk drive, and the like.

The comprehensive confidential information management and communication system 10 performs deep learning model analysis of each person's information, rules, and relationships 11. Such deep learning model analysis includes identification of changes of status or conditions, implementing updates to rules or relationships or other information, and causing the access controller 3 to notify professionals or other persons in accord with the rules. The deep learning model comprises a recurrent neural network configured to process time-series data. The model system maintains a feedback loop that adjusts model weights based on prediction accuracy over time.

The deep learning model analysis also identifies and flags any conflicting or contradicting information, rules, or relationships which might exist, and can identify potential future problems or potential advantageous changes which might be made.

The comprehensive confidential information management and communication system 10 performs a deep learning model analysis and update of outside rules and information 12. Information coming from outside the system can include such things as changes to the laws, rules, or regulations affecting estate planning or affecting relationships among persons and professionals, availability of new financial or investment products, or information about, for example, changes of status of certain professionals associated with one or more persons in the system. Information coming from outside can also include updated financial information such as updated interest rates, rates of return, forecasts, or warnings. Where a rule, relationship, or information of a person includes reference to some outside occurrence or condition, this deep learning model analysis and update of outside rules and information is the process by which the outside condition is flagged in the internal databases.

In use, the person authorizes use 31 of the comprehensive confidential information management and communication system 10 through the access controller 3. Sometimes the person might authorize use 31 of the system because a professional suggests 42 participation in the comprehensive confidential information management and communication system 10. The person then updates the rules-relationships database 1 information 32 and updates the information database 2 information 33. Such updating can include the initial entry of information or later updating in a repeating cycle 34 as appropriate. The updating is done through the access controller 3.

In use, after a professional initially registers 41 with the comprehensive confidential information management and communication system 10, and after the person identifies the professional and defines the relationship with the professional through an update 32 to the rules-relationships database 1, the professional can update 43 the persons information in the information database 2 and can access or receive relevant updated information 44 in accord with the relevant rules about the relationship in the rules-relationships database 1. The access control mechanism supports multi-factor authentication and biometric verification. The professional can make updates and access information on a repeating basis 45 as appropriate. The professional's access or receipt of information 44 can include secure push notifications of information flagged during deep learning model analysis of each person's information, rules, and relationships 11 and deep learning model analysis and update of outside rules and information 12, in order to ensure such flagged information is brought to the attention of the professional.

In use, the person or the person's designee or successor receives reports 35 from the comprehensive confidential information management and communication system 10 including reports about changed or refined predictions or projections, possibly based on new information, developed by the deep learning model continuous analysis and reporting where applicable 50.

In one aspect of the invention, the system 10 is configured to implement a method of training, by the deep learning processor 20, a network to analyze health information, rules and relationships data and update same on demand. The system 10 is configured to train, by the deep learning processor 20, a classification module to make predictions for each of multiple combinations of the health metrics, rules and relationships modules based on the learned features of the training data. The method of managing the comprehensive confidential data and communication includes training by the learning processor/system server a machine interpretable learning model to make predictions within the managing network based on the output of the deep learning continuous analysis of the collected health metrics, rules, and relationship data sets.

To generate an individual-specific report using the system of the present invention, the program directs the user to perform a series of steps, including:
a. authenticate a user via access controller 3;
b. gather individual-specific observations and instructions from internal and external sources;
c. update rules-relationship database and information database with new inputs;
d. apply deep learning model to analyze individual's current data, historical data from other individuals, and external rules and information;
e. generate predictive outcome and supporting rationale;
f. store prediction and rationale in model system for future refinement;
g. determine appropriate professional service providers based on prediction and current status;
h. securely transmit relevant updated information to selected professionals; and
i. log transmission for audit and compliance.

Aspects of the present invention are described herein with reference to a flowchart of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto the computer/processor 20, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the drawing illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). Each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Many other changes and modifications can be made in the system and method of the present invention without departing from the spirit thereof. I therefore pray that my rights to the present invention be limited only by the scope of the appended claims.

I claim:

1. A computer-implemented comprehensive confidential information management and communication system for managing and communicating confidential information pertinent to an individual's health, social, professional, and business relationships, and finances, the system comprising:
a secure data repository configured to store information and instructions relating to the individual's health, relationships, finances, and legal directives;
a data processing module configured to gather, maintain, update, and cross-reference said information and instructions from multiple sources;
a coordination engine configured to identify, and associate said information and instructions with a plurality of professional service providers, each having an independent relationship with the individual;
a condition monitoring module configured to detect changes in the individual's status or instructions;
a communication module configured to securely and timely transmit relevant information and instructions to appropriate professional service providers based on said detected changes; and
an access control mechanism configured to authenticate users and authorize access to specific subsets of said information based on predefined roles and relationships, wherein the data processing module comprises a deep-learning model trained to detect patterns and infer actionable insights from the stored information, the model being configured to adapt based on new data inputs and feedback from professional service providers.

2. The system of claim 1, wherein the data processing module utilizes machine learning algorithms to identify inconsistencies or gaps in the stored information.

3. The system of claim 1, wherein the coordination engine maintains a dynamic registry of professional service providers categorized by domain expertise and relationship history.

4. The system of claim 1, wherein the communication module supports encrypted messaging and audit trails for compliance with privacy regulations.

5. The system of claim 1, wherein the condition monitoring module integrates with external data sources including electronic health records, financial institutions, and legal document repositories.

6. The system of claim 1, wherein the access control mechanism supports multi-factor authentication and biometric verification.

7. The system of claim 1, further comprising an output module configured to present the prediction of the outcome to a user through a user interface.

8. The system of claim 7, wherein the output module is further configured to present an explanation of the prediction, including identifying the features that contributed most significantly to the prediction.

9. A computer-implemented method for managing and communicating confidential information pertinent to an individual's health, social, professional, and business relationships, and finances, comprising the steps of:
gathering multiple observations and instructions relating to the individual from various data sources;
analyzing said observations using a deep learning model trained on historical data from the individual and other individuals, the model configured to predict outcomes associated with changes in the individual's condition or directives;
substantially continuously updating the deep learning model with new observations and prediction outcomes to refine future predictions;
generating a prediction based on the analyzed observations and associating said prediction with supporting details;
adding said prediction and supporting details to the system for tracking and operational improvement;
determining appropriate professional service providers based on said prediction and the individual's current status; and
securely transmitting relevant information and instructions to said professional service providers in a timely manner.

10. The method of claim 9, wherein the deep learning model comprises a recurrent neural network configured to process time-series data.

11. The method of claim 9, wherein the model system maintains a feedback loop that adjusts model weights based on prediction accuracy over time.

12. The method of claim 9, wherein deep learning model analysis identifies and flags any conflicting or contradicting information, rules, or relationships which might exist, and identifies potential future problems or potential advantageous changes which might be made.

13. The method of claim 9, wherein the prediction and supporting details are stored in a secure audit log for compliance and review.

14. The method of claim 9, wherein the model system anonymizes data from other individuals used for training to preserve privacy.
